(19) United States
(12) Patent Application Publication
(10) Pub. No.: US 2024/0212474 A1
(43) Pub. Date: Jun. 27, 2024

(54) SYSTEM AND METHOD FOR A COMPUTERIZED EMERGENT RESPONSE ASSISTANCE DEVICE

(71) Applicant: GM GLOBAL TECHNOLOGY OPERATIONS LLC, Detroit, MI (US)

(72) Inventors: Gregg Prottengeier, Burton, MI (US); Luke J. Popiel, Oxford, MI (US); Russell A. Patenaude, Macomb Township, MI (US)

(73) Assignee: GM GLOBAL TECHNOLOGY OPERATIONS LLC, Detroit, MI (US)

(21) Appl. No.: 18/087,076

(22) Filed: Dec. 22, 2022

(51) Int. Cl. G08B 21/18

(52) U.S. Cl. CPC G08B 21/18

(57) ABSTRACT

A system for a computerized emergent response assistance device is provided. The system includes an emergent response device configured for assisting a user during an emergent condition. The system further includes a computerized emergent response assistance controller. The controller is configured for operating a setup routine digitally storing a location of the emergent response device and monitoring an input useful to identify occurrence of the emergent condition. The controller is further configured for analyzing the input to identify occurrence of the emergent condition and generating an alert to the user. The alert includes the location of the emergent response device.

### INTRODUCTION

The disclosure generally relates to a system and method for a computerized emergent response assistance device.

Emergencies take place in multiple contexts. A user may experience an emergent condition related to unintended combustion, allergies, or medical conditions such as heart issues and diabetes. Such emergent conditions may be sudden and unexpected. A user may panic, pre-existing emergent response device may be missing or poorly maintained, or the user may be in an unfamiliar environment.

### SUMMARY

A system for a computerized emergent response assistance device is provided. The system includes an emergent response device configured for assisting a user during an emergent condition. The system further includes a computerized emergent response assistance controller. The computerized emergent response assistance controller is configured for operating a setup routine digitally storing a location of the emergent response device and monitoring an input useful to identify occurrence of the emergent condition. The computerized emergent response assistance controller is further configured for analyzing the input to identify occurrence of the emergent condition and generating an alert to the user. The alert includes the location of the emergent response device.

In some embodiments, the emergent response device includes one of an extinguisher device or a defibrillator device.

In some embodiments, the emergent response device includes one of a bottle including medication or an epinephrine dispensing device.

In some embodiments, the emergent response device includes glucose useful to treat a diabetic emergency.

In some embodiments, monitoring the input includes monitoring a glucose level reading measured by a glucose level monitoring device.

In some embodiments, generating the alert includes providing intervention details stored within the computerized emergent response assistance controller.

In some embodiments, the computerized emergent response assistance controller is disposed within one of an emergent response electronic assistant unit or a smartphone device.

In some embodiments, the emergent response electronic assistant unit is disposed within one of a housing structure, an office building, a store, a school, or a hospital.

In some embodiments, the system further includes a smoke detector device. Monitoring the input useful to identify occurrence of the emergent condition includes monitoring an alert generated by the smoke detector device.

In some embodiments, the computerized emergent response assistance controller is disposed within a vehicle.

In some embodiments, the system further includes a temperature sensor within the vehicle. Monitoring the input useful to identify occurrence of the emergent condition includes monitoring data generated by the temperature sensor. Analyzing the input to identify occurrence of the emergent condition includes comparing the data generated by the temperature sensor to a threshold temperature.

In some embodiments, the system further includes an electronic device attached to the emergent response device useful to communicate with the computerized emergent response assistance controller and provide a location of the emergent response device to the computerized emergent response assistance controller.

In some embodiments, the electronic device includes one of a radio frequency identification chip, a low energy communications device, a circuit board, or a computerized device.

In some embodiments, the computerized emergent response assistance controller automatically updates the location of the emergent response device.

In some embodiments, the computerized emergent response assistance controller is further configured for providing a notification to the user when the emergent response device is moved from a registered location.

In some embodiments, the electronic device provides information related to usage of the emergent response device to the computerized emergent response assistance controller. The computerized emergent response assistance controller notifies a facilities manager when the emergent response device is utilized.

In some embodiments, the computerized emergent response assistance controller is further configured for storing an expiration date for the emergent response device and notifying the user of the expiration date.

In some embodiments, the computerized emergent response assistance controller is further configured for storing locations of a plurality of emergent response devices and directing the user to a closest of the plurality of emergent response devices.

According to one alternative embodiment, a system for a computerized emergent response assistance device is provided. The system includes a vehicle. The vehicle includes an emergent response device configured for assisting a user during an emergent condition and a computerized emergent response assistance controller. The computerized emergent response assistance controller is configured for operating a setup routine digitally storing a location of the emergent response device and monitoring an input useful to identify occurrence of the emergent condition. The computerized emergent response assistance controller is further configured for analyzing the input to identify occurrence of the emergent condition and generating an alert to the user. The alert includes the location of the emergent response device. The vehicle further includes an electronic device attached to the emergent response device useful to communicate with the computerized emergent response assistance controller and provide a location of the emergent response device to the computerized emergent response assistance controller.

According to one alternative embodiment, a method for operating a computerized emergent response assistance device is provided. The method includes, within a computerized processor, operating a setup routine digitally storing a location of an emergent response device and monitoring an input useful to identify occurrence of the emergent condition. The method further includes, within the computerized processor, analyzing the input to identify occurrence of the emergent condition and generating an alert to a user, wherein the alert includes the location of the emergent response device.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 schematically illustrates an exemplary system for a computerized emergent response assistance device, in accordance with the present disclosure;

FIG. 2 schematically illustrates an alternative exemplary system for a computerized emergent response assistance device, in accordance with the present disclosure;

FIG. 3 schematically illustrates an alternative exemplary system for a computerized emergent response assistance device, in accordance with the present disclosure;

FIG. 4 is a flowchart illustrating a method for configuring a computerized emergent response assistance device, in accordance with the present disclosure;

FIG. 5 schematically illustrates the computerized emergent response assistance controller, in accordance with the present disclosure;

FIG. 6 schematically illustrates an emergent response device embodied as a medication bottle, in accordance with the present disclosure;

FIG. 7 schematically illustrates an emergent response device embodied as an epinephrine dispensing device, in accordance with the present disclosure;

FIG. 8 schematically illustrates an emergent response device embodied as an extinguisher device, in accordance with the present disclosure;

FIG. 9 schematically illustrates an emergent response device embodied as an AED device, in accordance with the present disclosure; and

FIG. 10 is a flowchart illustrating a method to operate a computerized emergent response assistance device, in accordance with the present disclosure.

### DETAILED DESCRIPTION

Emergent conditions may occur unexpectedly. A user may experience a condition that requires immediate access to emergency equipment. A user may panic and fail to remember where emergency equipment is stored as a result of the panic. A user may attempt to access the emergency equipment and find that the equipment is not charged or has not been properly maintained. A user may be in an unfamiliar environment such as in a friend's house or in a rental vehicle.

A computerized emergent response assistance device is provided. The disclosed computerized emergent response assistance device enables a user/operator to register local emergent response device prior to existence of an emergent condition. The computerized emergent response assistance device may be programmed with a setup routine, requesting that the user/operator provide useful information related to each emergent response device. For example, the computerized emergent response assistance device may request information related to a location and an expiration date for each emergent response device being registered. The computerized emergent response assistance device may be utilized in a wide variety of environments, including but not limited to within a home or an apartment, within a vehicle, within a boat, within an airplane, within a business office, within a sports arena, within a hospital or medical facility, in a public part, within a school, or within other similar settings.

Emergent response devices may be unconnected devices. Examples may include an extinguisher device without electronics built into it or an unimproved plastic bottle of medicine prescribed for a user. The computerized emergent response assistance device may initially request information about the unconnected devices, such as identity or description, location at time of registry, specific user information such as whom has the medication been prescribed for, expiration date, instructions for use, and contact information for repurchase, refill, recharge, or maintenance.

Emergent response devices may include a computerized controller upon or integrated with the devices. For example, a defibrillator (AED) may include a computerized controller useful to automatically provide life-saving electrical signals to a person having a heart incident. The computerized controller may include a communications module such as a wireless communications device useful to connect to a local WIFI network. The AED may include programming to provide information about the AED to the computerized emergent response assistance device, for example, including location, maintenance status, power or charge status, and an alert for activation or removal from a storage case.

Emergent devices may include limited electronics installed thereto, providing limited information such as a proximity to a WIFI network router or smartphone device. In one example, an extinguisher device may include a radio frequency identification (RFID) chip. The computerized emergent response assistance device may utilize a localized wireless device such as a WIFI network router or a smartphone device to periodically monitor information available from the RFID chip and save that information for emergent access. In one embodiment, a storage location may be initially registered, and movement of the RFID chip from the storage location may generate an alert based upon the extinguisher device potentially being out of an expected location or low on chemical charge due to usage. A device that is frequently moved or packed in luggage, such as an epinephrine dispensing device, may similarly include an RFID chip useful to enable tracking and rapid location of the epinephrine dispensing device, helping a user to determine where she or he last had the device.

The disclosed system and method may be employed through a physical device mounted upon a wall or within a computerized module installed to a vehicle. In another embodiment, the disclosed system and method may be operated within a remote server device, and local computerized devices such as smartphones and wirelessly connected electronic devices may be utilized to collect information to diagnose an emergent condition and further to provide information to users in proximity to the emergent condition. The disclosed system and method may include use or availability of a smartphone software application that may be widely distributed, such that a plurality of users may install the software application upon their smartphones. In one embodiment, a cellular operating system, a phone carrier, large retail stores, etc. may embed the functionality of the disclosed software application in other software. For example, a grocery store chain may market and make available a software application providing coupons for use in the store, and part of the programming of that software application may include the functionality of the disclosed system and method, providing customers important information when the customers are within a store operated by the grocery store chain.

Emergent devices, such as a glucose monitoring/diabetes intervention device may report readings taken by a patient to the computerized emergent response assistance device. Intervention settings may be programmed into the computerized emergent response assistance device, such that if glucose readings are above or below an acceptable range, the computerized emergent response assistance device may activate programming to prompt action from the patient, notify a caretaker, or contact emergency response personnel.

Occurrence of an emergent condition may be input by a user, for example, through a smartphone. A user may have a software application installed to a smartphone, and the smartphone may be configured for monitoring auditory signals, for example, to determine occurrence of an excited exclamation which may indicate unintended combustion. In another example, the smartphone may be activated by a durable graphical icon displayed upon the touchscreen of the device, and, once activated, the user may be presented with a plurality of selectable choices upon the touchscreen. In one example, the touchscreen may display selectable options enabling a user to quickly indicate a need for an extinguisher device, an epinephrine dispensing device, or first aid kit. The touchscreen may additionally display a selectable option to contact emergency responders.

Occurrence of an emergent condition may be determined based upon data gathered by one or more sensors. In one embodiment, the computerized emergent response assistance device may be in communication with a smoke detector device or with a temperature sensor. Data from the connected device, for example, compared to a threshold temperature value, may indicate potential occurrence of unintended combustion. The computerized emergent response assistance device may be in communication with a glucose level measuring device assisting a user with diabetes to monitor blood sugar levels. Data from the connected glucose level measuring device may indicate a need for medical intervention. The computerized emergent response assistance device may be in communication with a smartphone device. The smartphone device may include a selectable software application which may utilize voice recognition to determine occurrence of an emergent condition or may utilize connectivity and diagnostics available through the smartphone device to indicate an emergent condition.

Upon determining or monitoring occurrence of an emergent condition, the computerized emergent response assistance device may command or initiate a number of responses. A localized alert may be activated, for example, with a speaker device being used to provide audible information to persons present within the house, vehicle, office, etc. wherein the emergent condition is identified. The computerized emergent response assistance device may contact a user and provide information to the user. For example, a user or a group of users may be contacted through a smartphone.

The computerized emergent response assistance device, upon occurrence of an emergent condition, may contact the user that setup or registered the computerized emergent response assistance device. In another embodiment, a group of users may be contacted upon occurrent of an emergent condition. A group of users to be contacted may be defined by a user, for example, including a family of users, a group of roommates living in a same household, a group of employees that work in a particular office building, or other user defined group. In another embodiment, a group of users may be defined geographically or by proximity, for example, with users present within a house with the application installed to their smartphone being defined as a group. In another embodiment, users present within a vehicle with the application installed to their smartphone may be defined as a group. In one embodiment, a building, a vehicle, or a locality may be registered as an open to non-specific users zone. In such an open to non-specific users zone, users with the application installed to a smartphone determined to be within the zone may receive notifications.

In addition to alerts generated related to an identified emergent condition, other informational notices may be generated. For example, if a medication is determined to have expired, a notification may be generated recommending replacement of the medication. In another example, if an extinguisher device is determined to be low on charge or to be placed out of its normal location, a notification may be generated. In another example, if an AED or a fire extinguisher is removed from its storage location, a notification may be generated. In one embodiment, a first list of users to be notified in case of an emergent condition, for example, users within a proximity that may need to know where an emergent response device is stored, may be defined, and a second list of users to be notified in case of a maintenance or expiration date issue may additionally be defined

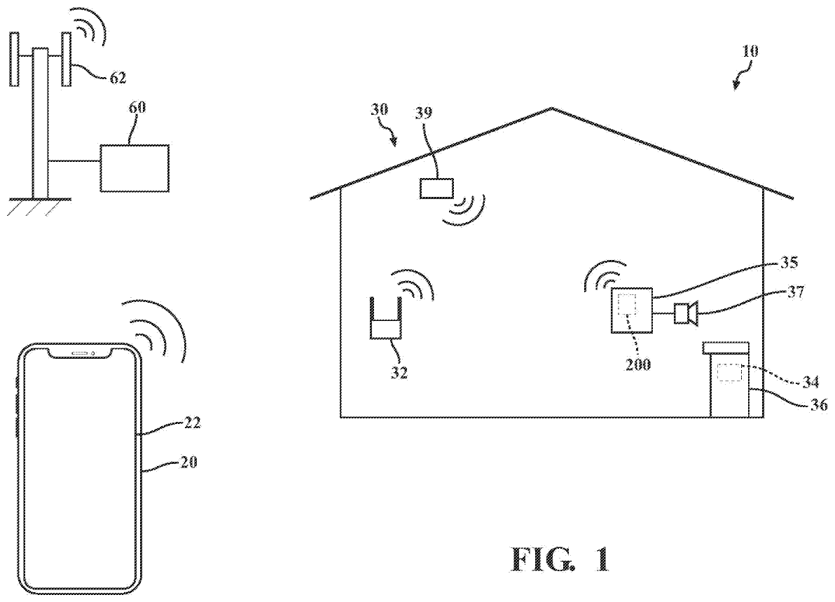

Referring now to the drawings, wherein like reference numbers refer to like features throughout the several views, FIG. 1 schematically illustrates an exemplary system 10 for a computerized emergent response assistance device. The system 10 is illustrated including a housing structure 30. The housing structure 30 includes an emergent response electronic assistant unit 35 including a computerized emergent response assistance controller 200 and an audio speaker 37. The emergent response electric assistant unit 35 is illustrated as a wall mounted unit, while it may alternatively be embodied as a tabletop unit or other similar electronic device. In one embodiment, the disclosed system and process may be operated within a smartphone device 20. The emergent response electronic assistant unit 35 may further include a microphone device useful to monitor audio information within the housing structure 30. The housing structure 30 is further illustrated including an optional WIFI router 32 and an optional smoke detector 39. An exemplary first aid kit 34 is illustrated contained within a cabinet 36.

The system 10 is further illustrated including an optional smartphone device 20 including a touchscreen display 22 and an optional remote server device 60 in communication with other components of the system 10 through a cellular communications network 62.

The computerized emergent response assistance controller 200 may be initially configured to store information related to emergent response devices within the housing structure 30. A user may register a location of the first aid kit 34 as being stored within the cabinet 36.

The computerized emergent response assistance controller 200 monitors information from available input devices and identifies occurrence of an emergent condition within the housing structure 30. If an emergent condition is identified, the computerized emergent response assistance controller 200 may issue an alert. In one embodiment, the computerized emergent response assistance controller 200 may monitor through the microphone device a statement “Where is the first aid kit?” This stated need for the first aid kit may be identified as an emergent condition. In response to the emergent condition, the computerized emergent response assistance controller 200 may issue an audible notification, “The first aid kit is stored within the cabinet.” In some embodiments, the notification may further be provided upon the smartphone device 20.

In some embodiments, information such as usage and purchase date of the first aid kit 34 may be monitored and analyzed. The remote server device 60 may include programming to track information about the first aid kit 34 and prompt a user through the smartphone device 20 whether a replacement first aid kit should be ordered. Once a new first aid kit 34 is delivered to the housing structure 30, the computerized emergent response assistance controller 200 may prompt the user through the smartphone device 20 to confirm that the new first aid kit 34 has been placed within the cabinet 36.

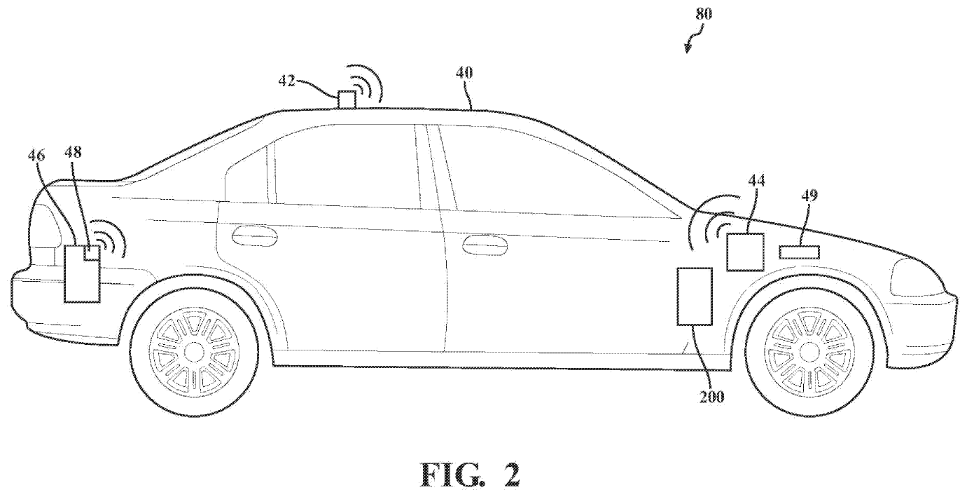

FIG. 2 schematically illustrates an alternative exemplary system 80 for a computerized emergent response assistance device. The system 80 is illustrated including a vehicle 40 including the computerized emergent response assistance controller 200 installed thereto. The vehicle 40 is further illustrated including a local wireless communication device 44 and a cellular communication device 42. The vehicle 40 is illustrated including an emergent response device 46 provided within a rear storage area of the vehicle 40. The emergent response device 46 is illustrated including an RFID chip 48 affixed thereto. The emergent response device 46 may include an extinguisher device an AED device, an epinephrine dispensing device, or other similar device. The RFID chip 48 is configured to provide information to the local wireless communication device 44.

The computerized emergent response assistance controller 200 is configured to monitor inputs available within the vehicle 40 and identify occurrence of an emergent condition. In one embodiment, the vehicle 40 is equipped with a temperature sensor 49. The temperature sensor 49 may be configured for providing temperature data within a sensitive area of the vehicle 40 to the computerized emergent response assistance controller 200. If the temperature data indicates that an unintended combustion event may be occurring within the vehicle 40, the computerized emergent response assistance controller 200 may notify users occupying the vehicle 40. The notification may include a location of the emergent response device 46 including an exemplary extinguisher device within the vehicle 40. The notification may further include a recommendation that the vehicle 40 be immediately parked. The notification may further include communication over the cellular communication device 42 to local emergency responders.

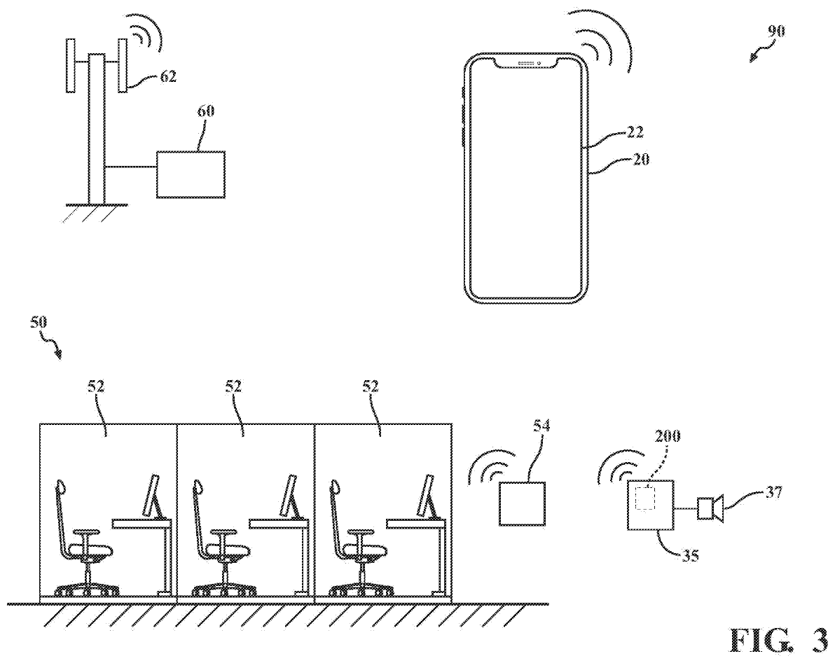

FIG. 3 schematically illustrates an alternative exemplary system 90 for a computerized emergent response assistance device. The system 90 is illustrated including an office environment 50 including a plurality of office worker stations 52, a wall mounted emergent response device 54 including an exemplary AED device. The emergent response electronic assistant unit 35 including the computerized emergent response assistance controller 200 and the audio speaker 37 is illustrated. The speaker 37 is provided as one way in which audio messages may be provided within the office environment 50. Audible outputs may be provided through a sound system that may be heard throughout the building or office environment 50. The computerized emergent response assistance controller 200 may include a cellular communication capability enabling the computerized emergent response assistance controller 200 to send and receive information with the remote server device 60 through the cellular communications network 62. The computerized emergent response assistance controller 200 may communicate with the smartphone device 20 including the touchscreen display 22 such that users within the office environment 50 may be aware of an identified emergent condition. In one embodiment, the computerized emergent response assistance controller 200 may provide a notice to a facilities manager for the office environment 50, enabling the facilities manager to react to the identified emergent condition, for example, giving the facilities manager a prompt on the smartphone 20 to unlock security doors such that emergency response personnel may quickly access the building.

FIGS. 1-3 are provided illustrating exemplary environments in which the computerized emergent response assistance controller 200 may be utilized. These examples are not intended to be limiting, for example, additionally including a boat, where users upon the boat may be informed during an identified emergent condition where lifejackets may be found or where an emergency flare is kept. In another example, the computerized emergent response assistance controller 200 may be utilized within a grocery store, wherein customers within the store with the application installed to their smartphones may be informed during an unintended combustion event where the emergency exits are located. Information may be shared between environments, for example, with a medical emergent condition identified in the housing structure 30 of FIG. 1 being shared with a registered caretaker within the vehicle 40 of FIG. 2 through cellular communications enabled by the cellular communications network 62 of FIG. 1.

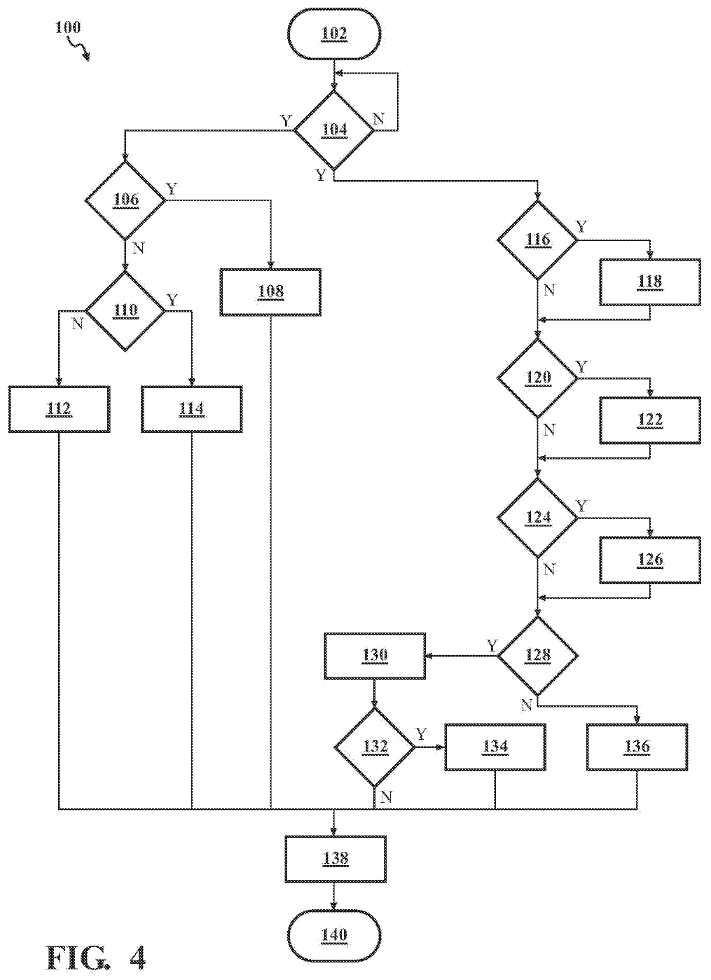

FIG. 4 is a flowchart illustrating a method 100 for configuring a computerized emergent response assistance device. The disclosed method 100 may be described as a setup routine useful to digitally store a location of an emergent response device. The disclosed method 100 is illustrated being operated within the computerized emergent response assistance controller 200 of FIG. 1, although the method 100 may be operated in other ways, for example, as a software application operated within the remote server device 60 of FIG. 1 or as a software application operated within the smartphone device 20 of FIG. 1. The method 100 starts with step 102. At step 104, a determination is made whether a routine to setup the computerized emergent response assistance device is to be operated. If the routine to setup the assistant is not to be operated, the method 100 returns to step 104 to continue monitoring for the routine to be activated. If the routine is to setup the computerized emergent response assistance device is to be operated, then the method 100 may advance to step 106, step 116, or steps 106 and 116 in parallel.

Starting at step 106, a plurality of method steps is disclosed related to utilizing electronic devices to register or update a location of an emergent response device. At the step 106, a determination is made whether a registered emergent response device is available within a short-range communications network. If the registered emergent response device is available within the short-range communications network, the method 100 advances to the step 108, wherein the computerized emergent response assistance controller 200 of FIG. 1 stores or updates the precise location of the registered emergent response device. The method 100 then advances to the step 138. The step 106 may include making the disclosed determination regarding a plurality or a list of registered emergent response devices previously setup within the computerized emergent response assistance controller 200. The step 106 may additionally search for previously unregistered emergent response devices that are listed upon an approved list of automatically registerable emergent response devices established by the publisher of the software of the computerized emergent response assistance controller 200. For a registered emergent response device that is not detected in communication with the short-range communications network, the method 100 advances to the step 110, wherein a determination is made whether the user operating the method 100 is utilizing an electronic device such as the smartphone device 20 of FIG. 1 which may register a location. If the user is utilizing the electronic device which may register a location, the method 100 advances to the step 114, where the user is prompted to physically move to the location where the emergent response device is located, and, upon monitoring the user making an indication, for example, to the touchscreen display 22, the location of the emergent response device may be stored based upon the location of the electronic device being operated by the user. The method 100 then advances to the step 138. If the user is not utilizing an electronic device which may register a location, the method 100 advances to the step 112 where the user is requested to manually input a location for the emergent response device. The method 100 then advances to the step 138.

Starting at step 116, a plurality of method steps is disclosed related to a user manually selecting and registering a location of an emergent response device. At step 116, the user is provided the option to add an extinguisher device. If the user elects to register an extinguisher device, the method 100 advances to the step 118, where the computerized emergent response assistance controller 200 stores a location of the extinguisher device and stores an expiration date for the extinguisher device. The method 100 then advances to step 120. If the user does not elect to register an extinguisher device, the method 100 advances to the step 120. At step 120, the user is provided the option to add a defibrillator device. If the user elects to register a defibrillator device, the method 100 advances to the step 122, where the computerized emergent response assistance controller 200 stores a location of the defibrillator device and stores an expiration date for the defibrillator device. The method 100 then advances to step 124. If the user does not elect to register a defibrillator device, the method 100 advances to the step 124. At step 124, the user is provided the option to add an epinephrine dispensing device. If the user elects to register an epinephrine dispensing device, the method 100 advances to the step 126, where the computerized emergent response assistance controller 200 stores a location of the epinephrine dispensing device and stores an expiration date for the epinephrine dispensing device. The method 100 then advances to step 128. If the user does not elect to register an epinephrine dispensing device, the method 100 advances to the step 128.

At step 128, the user is provided the option to add a glucose and/or medication configured for aiding in a diabetic emergency. If the user elects to register the glucose and/or the medication, the method 100 advances to the step 130, where the computerized emergent response assistance controller 200 stores a location of the glucose and/or medication and stores an expiration date for the glucose and/or medication. The method 100 then advances to step 132. If the user does not elect to register the glucose and/or medication, the method 100 advances to the step 138. At step 132, the user is prompted to enter medical intervention settings. If the user elects to enter medical intervention settings, the method 100 advances to step 134, where the user may enter a variety of intervention settings including prompts to take medication, prompts to contact a caretaker, and prompts to contact emergency responders. Step 134 may further include electronic interaction between the computerized emergent response assistance controller 200 and a glucose level monitoring device. The method 100 then advances to the step 138. If the user does not elect to enter medical intervention settings, the method advances to step 138.

At step 128, if the user does not elect to register the glucose and/or the medication, the method 100 advances to the step 136. If no emergent response device was added by the method 100, the computerized emergent response assistance controller 200 may report failure to record a device locations to the user and provide the user with an option to restart the process. The method 100 may then advance to the step 138. At step 138, the computerized emergent response assistance controller 200 stores the relevant information gathered within the method 100 for incident use. The method 100 ends at step 140. The method 100 is an example of a method or process for configuring a computerized emergent response assistance device. The steps of the method 100 are intended to be non-limiting, additional and/or alternative method steps are envisioned, and the disclosure is not intended to be limited to the examples provided herein.

FIG. 5 schematically illustrates the computerized emergent response assistance controller 200. Computerized emergent response assistance controller 200 includes processing device 210, communications device 220, data input output device 230, and memory storage device 240. It is noted that computerized emergent response assistance controller 200 may include other components and some of the components are not present in some embodiments. The disclosed computerized emergent response assistance controller 200 may be located within a dedicated electronic device that may be wall mounted or situated upon an exemplary tabletop. Alternatively, the computerized emergent response assistance controller 200 may be operated within the smartphone device 20 of FIG. 1

The processing device 210 may include memory, e.g., read only memory (ROM) and random-access memory (RAM), storing processor-executable instructions and one or more processors that execute the processor-executable instructions. In embodiments where the processing device 210 includes two or more processors, the processors may operate in a parallel or distributed manner. Processing device 210 may execute the operating system of the computerized emergent response assistance controller 200. Processing device 210 may include one or more modules executing programmed code or computerized processes or methods including executable steps. Illustrated modules may include a single physical device or functionality spanning multiple physical devices. In the illustrative embodiment, the processing device 210 also includes an information collection module 212, an emergent condition identification module 214, and an alert/reaction module 216, which are described in greater detail below.

The data input output device 230 is a device that is operable to take data gathered from sensors and devices throughout the vehicle and process the data into formats readily usable by processing device 210. Data input output device 230 is further operable to process output from processing device 210 and enable use of that output by other devices or control modules throughout the vehicle.

The communications device 220 may include a communications/data connection with a bus device configured to transfer data to different components of the system and may include one or more wireless transceivers for performing wireless communication.

The memory storage device 240 is a device that stores data generated or received by the computerized emergent response assistance controller 200. The memory storage device 240 may include, and is not limited to, a hard disc drive, an optical disc drive, and/or a flash memory drive.

The information collection module 212 may collect data from a plurality of sources useful to determine occurrence of an emergent event. Sources may include the smartphone device 20 of FIG. 1, a microphone device such as the microphone device built into the emergent response electronic assistant unit 35, the smoke detector device 39 of FIG. 1, the temperature sensor 49 of FIG. 2, and/or the emergent condition device 54 of FIG. 3. Speech, a sound of an alarm going off, a data indication of an alert or a temperature violation, or other similar data may be collected and provided to the emergent condition identification module 214.

The emergent condition identification module 214 receives data from the information collection module 212. The emergent condition identification module 214 analyzes data from available sources to diagnose an occurrence of or a threshold indication of an emergent event. The emergent condition identification module 214 may further monitor information such as expiration dates of emergent response devices and may indicate recommended refills and replacements based upon the information.

The alert/reaction module 216 may receive an indication from the emergent condition identification module 214 and may take action based upon the indication. For example, the alert/reaction module 216 may issue an audible alert to a proximity associated with the identified emergent condition, may provide information to relevant users related to a location of a related emergent response device, and may provide information over a wireless communication network to relevant persons and/or authorities. In one embodiment, the alert/reaction module 216 may include programming to determine which of a plurality of emergent response device is closest to a user and provide instructions for the user on how to get to that closest device. The alert/reaction module 216 may further receive alerts related to expiration dates from the emergent condition identification module 214 and may provide notices to a user recommending actions such as ordering refills and replacements.

Computerized emergent response assistance controller 200 is provided as an exemplary computerized device capable of executing programmed code to accomplish the methods and processes described herein. A number of different embodiments of computerized emergent response assistance controller 200, devices attached thereto, and modules operable therein are envisioned, and the disclosure is not intended to be limited to examples provided herein.

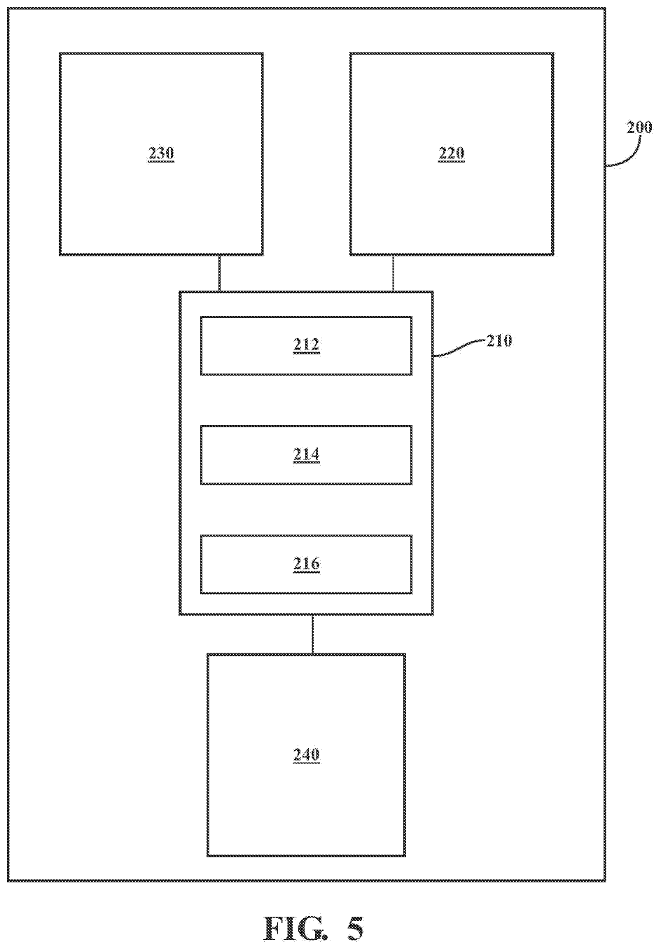

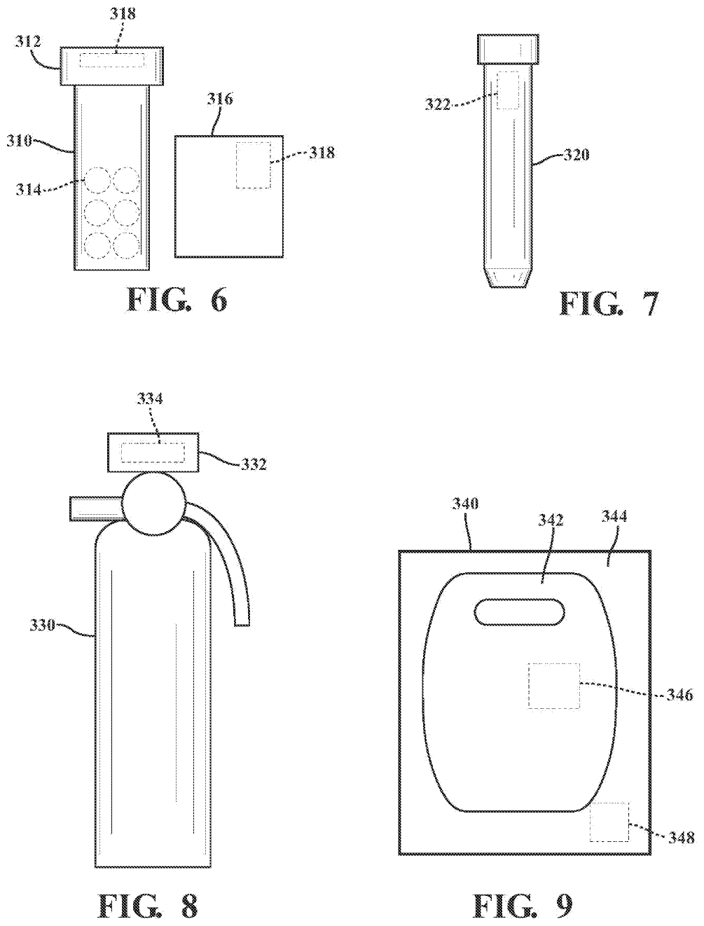

FIG. 6 schematically illustrates an emergent response device 310 embodied as a medication bottle. The emergent response device 310 is illustrated including a plurality of medical doses 314 contained therewithin, a label 316, and a removeable cap 312 secured to the emergent response device 310. In one embodiment, the medical doses 314 may be glucose or a medicine useful during a diabetic emergency. The label 316 is illustrated detached from the emergent response device 310 and may include adhesive used to wrap and adhere the label to the emergent response device 310. Exemplary RFID chips 318 are illustrated installed to the cap 312 and/or the label 316. The RFID chip 318 may be useful to providing a location of the emergent response device 310 to the computerized emergent response assistance controller 200 of FIG. 1. In another embodiment, the RFID chip 318 may alternatively be a low energy communications device capable of providing a location.

FIG. 7 schematically illustrates an emergent response device 320 embodied as an epinephrine dispensing device. The emergent response device 320 is illustrated including an exemplary circuit board 322 installed thereto. The circuit board 322 may be useful to providing a location of the emergent response device 320 to the computerized emergent response assistance controller 200 of FIG. 1.

FIG. 8 schematically illustrates an emergent response device 330 embodied as an extinguisher device. The emergent response device 330 is illustrated including an exemplary electronics module 332 including a microprocessor control unit 334 installed thereto. The microprocessor control unit 334 may be useful to providing a location of the emergent response device 330 to the computerized emergent response assistance controller 200 of FIG. 1.

FIG. 9 schematically illustrates an emergent response device 340 embodied as an AED device. The emergent response device 340 is illustrated including a removeable AED unit 342 useful to treat a patient and a wall-mounted base station 344. The AED unit 342 may include a computerized module 346 useful to control operation of the medical functions of the AED unit 342. The computerized module 346 may further utilize wireless communications to provide information such as location, usage, patient information, and charge level to the computerized emergent response assistance controller 200 of FIG. 1. The wall-mounted base station 344 may include a computerized module 348 useful to monitor removal of the AED unit 342. The computerized module 348 may further utilize wireless communications to provide information such as location and removal of the AED unit to the computerized emergent response assistance controller 200 of FIG. 1.

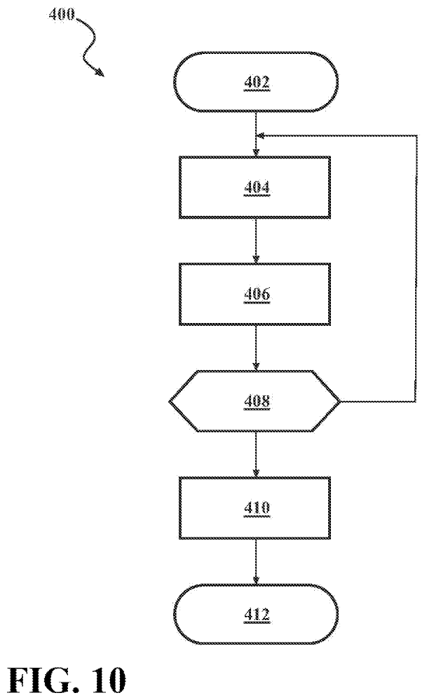

FIG. 10 is a flowchart illustrating a method 400 to operate a computerized emergent response assistance device. The method 400 is described as being utilized with the system 10 of FIG. 1, however, the method 400 may be utilized with other embodiments of the system. The method 400 starts at step 402. At step 404, inputs related to environmental or contextual conditions that may indicate occurrence of an emergent event are monitored. At step 406, information from the monitored inputs is analyzed to determine occurrence of an emergent event or condition. At step 408, a determination is made whether the emergent event or condition is identified at step 406. If the emergent event or condition is identified, then the method 400 advances to step 410. If the emergent event or condition is not identified, the method 400 returns to the step 404, where the inputs are iteratively monitored. At step 410, an alert is generated and information may be relayed to one or more users. In one embodiment, the information relayed to the one or more users may include a location of an emergent response device useful in light of the identified emergent event or condition. At step 412, the method 400 ends. The method 400 is an exemplary method or process for operation of a computerized emergent response assistance device, a number of additional and/or alternative method steps are envisioned, and the disclosure is not intended to be limited to the examples provided herein.

While the best modes for carrying out the disclosure have been described in detail, those familiar with the art to which this disclosure relates will recognize various alternative designs and embodiments for practicing the disclosure within the scope of the appended claims.

What is claimed is:

1. A system for a computerized emergent response assistance device, the system comprising:
an emergent response device configured for assisting a user during an emergent condition; and
a computerized emergent response assistance controller, configured for:
operating a setup routine digitally storing a location of the emergent response device;
monitoring an input useful to identify occurrence of the emergent condition;
analyzing the input to identify occurrence of the emergent condition; and
generating an alert to the user, wherein the alert includes the location of the emergent response device.

2. The system of claim 1, wherein the emergent response device includes one of an extinguisher device or a defibrillator device.

3. The system of claim 1, wherein the emergent response device includes one of a bottle including medication or an epinephrine dispensing device.

4. The system of claim 1, wherein the emergent response device includes glucose useful to treat a diabetic emergency.

5. The system of claim 4, wherein monitoring the input includes monitoring a glucose level reading measured by a glucose level monitoring device.

6. The system of claim 4, wherein generating the alert includes providing intervention details stored within the computerized emergent response assistance controller.

7. The system of claim 1, wherein the computerized emergent response assistance controller is disposed within one of an emergent response electronic assistant unit or a smartphone device.

8. The system of claim 7, wherein the emergent response electronic assistant unit is disposed within one of a housing structure, an office building, a store, a school, or a hospital.

9. The system of claim 7, further including a smoke detector device; and
wherein monitoring the input useful to identify occurrence of the emergent condition includes monitoring an alert generated by the smoke detector device.

10. The system of claim 1, wherein the computerized emergent response assistance controller is disposed within a vehicle.

11. The system of claim 10, further comprising a temperature sensor within the vehicle;
wherein monitoring the input useful to identify occurrence of the emergent condition includes monitoring data generated by the temperature sensor; and
wherein analyzing the input to identify occurrence of the emergent condition includes comparing the data generated by the temperature sensor to a threshold temperature.

12. The system of claim 1, further comprising an electronic device attached to the emergent response device useful to communicate with the computerized emergent response assistance controller and provide a location of the emergent response device to the computerized emergent response assistance controller.

13. The system of claim 12, wherein the electronic device includes one of a radio frequency identification chip, a low energy communications device, a circuit board, or a computerized device.

14. The system of claim 12, wherein the computerized emergent response assistance controller automatically updates the location of the emergent response device.

15. The system of claim 14, wherein the computerized emergent response assistance controller is further configured for providing a notification to the user when the emergent response device is moved from a registered location.

16. The system of claim 12, wherein the electronic device provides information related to usage of the emergent response device to the computerized emergent response assistance controller; and
wherein the computerized emergent response assistance controller notifies a facilities manager when the emergent response device is utilized.

17. The system of claim 1, wherein the computerized emergent response assistance controller is further configured for:
storing an expiration date for the emergent response device; and
notifying the user of the expiration date.

18. The system of claim 1, wherein the computerized emergent response assistance controller is further configured for:
storing locations of a plurality of emergent response devices; and
directing the user to a closest of the plurality of emergent response devices.

19. A system for a computerized emergent response assistance device, the system comprising:
a vehicle, including:
an emergent response device configured for assisting a user during an emergent condition;
a computerized emergent response assistance controller, configured for:
operating a setup routine digitally storing a location of the emergent response device;
monitoring an input useful to identify occurrence of the emergent condition;
analyzing the input to identify occurrence of the emergent condition; and
generating an alert to the user, wherein the alert includes the location of the emergent response device; and
an electronic device attached to the emergent response device useful to communicate with the computerized emergent response assistance controller and provide a location of the emergent response device to the computerized emergent response assistance controller.

20. A method for operating a computerized emergent response assistance device, the method comprising:
within a computerized processor,
operating a setup routine digitally storing a location of an emergent response device;
monitoring an input useful to identify occurrence of an emergent condition;
analyzing the input to identify occurrence of the emergent condition; and
generating an alert to a user, wherein the alert includes the location of the emergent response device.
